(19) United States
(12) Patent Application Publication
(10) Pub. No.: US 2004/0229246 A1
(43) Pub. Date: Nov. 18, 2004

(54) Diagnostic markers for therapeutic treatment

(75) Inventors: Pnina Fishman, Herzliya (IL); Lea Madi, Rishon Le Zion (IL); Sara Bar Yahuda, Rishon Le Zion (IL)

Correspondence Address: BROWDY AND NEIMARK, P.L.L.C., 624 NINTH STREET, NW, SUITE 300, WASHINGTON, DC, 20001-5303, US

(73) Assignee: Can-Fite Biopharam Ltd., Petach Tikva (IL)

(21) Appl. No.: 10/763,190

(22) Filed: Jan. 26, 2004

Related U.S. Application Data
(63) Continuation-in-part of application No. 10/689,508, filed on Oct. 21, 2003.
(60) Provisional application No. 60/419,595, filed on Oct. 21, 2002.

(51) Int. Cl. C12Q 1/68; G01N 33/53; G01N 33/567

(52) U.S. Cl. 435/6; 435/7.2

(57) ABSTRACT

A method for monitoring the effectiveness of an administered agent that interacts with the A₃ adenosine receptor (A3AR) in treatment of a disease state in an individual is provided. The level of at least one parameter of a biological marker is determined and compared to a control level, the control level being the level of the parameter without treatment, which is determined either as the level of the parameter in the individual prior to treatment or the level of the parameter in untreated control subject having the disease state. Monitoring a change in these parameters versus control, may also be used for screening agents that interact with the A3AR for such that can be used for a treating a disease state.

### FIELD OF THE INVENTION

This invention relates to the field of diagnosis and in particular to biological markers associated with disease states, and associated with therapeutic treatment.

### BACKGROUND OF THE INVENTION

A₃ adenosine receptors belong to the family of the Gi-protein associated cell surface receptors. Receptor activation leads to its internalization and the subsequent inhibition of adenylate cyclase activity, cAMP formation and protein kinase A (PKA) expression, resulting in the initiation of various signaling pathways (Olah et al, 2000; Poulsen et al, 1998). PKA contains a catalytic subunit PKAc which dissociates from the parent molecule upon activation with cAMP. Recent studies have demonstrated that PKAc phosphorylates and inactivates a GSK-3β (Fang et al, 2000).

Recently, it has been shown that 1-deoxy-1-[6[[(3-iodophenyl)methyl]amino]-9H-purine-9-yl]-N-methyl-β-D-ribofura-nuronaminde (IB-MECA), a stable agonist to A3AR, alters the expression of GSK-3β and β-catenin, key components of the Wnt signaling pathway. Consequently it leads to the inhibition of the expression of the cell cycle progression genes, c-myc and cyclin D1 (Fishman et al, 2002).

### SUMMARY OF THE INVENTION

The present invention is based on the finding that agonists of the A₃ adenosine receptor (A3AR) alter several characteristics of cellular markers, including their expression level, phosphorylation, and their cellular localization. Thus it is possible to assess, or monitor the success of a therapeutic treatment by A3AR modulators by monitoring the change of the expression, phosphorylation, or localization of a number of biological markers.

Thus, the present invention concerns a method for monitoring the effectiveness of an administered agent that interacts with the A3AR in treatment of a disease state in an individual, by monitoring, continuously or at one or more pre-defined time points, the level of at least one parameter of a biological marker as compared to a control level, the control level being the level of the parameter without treatment, which is determined either as the level of the parameter in the individual prior to treatment or the level of the parameter in an untreated control subject having the disease state. The treatment consists of administration to the individual of an agent that interacts with the A3AR. The method comprises:

(i) withdrawing a sample of cells associated with the disease state or of tissue containing said cells from an individual at a pre-defined time point following administration of the agent to the individual, the time point being selected so as to permit the agent to reach and affect said cells;

(ii) detecting the level of at least one physiological parameter of at least one biological marker in said cells, the marker being an A3AR, or an element associated with an A3AR signal transduction pathway downstream to A3AR; and

(iii) comparing the level of said at least one parameter to a control level, the control level being the level thereof in such cells or tissue from the same individual before administration of said agent, or being a standard reference for said marker which is indicative of an untreated disease state;

wherein a difference in level of the physiological parameter between the treated cells and the control being indicative of the effectiveness of said treatment of the disease state.

The agent may be one or more drugs that exert an agonistic or antagonistic effect on the A3AR. The agent being an A3AR agonist, such as IB-MECA, Cl-IB-MECA and others, is a preferred embodiment of the invention. The agent is typically a single drug with an agonistic or antagonistic effect on the A3AR although it may at times consist of two or more drugs administered in combination, for example an agonist and a modulator.

The term “biological marker” or, in short, “marker” according to the invention should be construed in its broadest sense as referring to any endogenous, cell associated, substance, including, without being limited thereto, an amino acid comprising compound (e.g., protein, polypeptide or peptide) nucleic acid compound (e.g., mRNA), a cell metabolite, which is either present at the surface of the diseased cells (e.g., cell surface receptor, cell surface glycoproteins, etc.), shed or secreted from the cell or which is present within the,cell (e.g., a kinase). In particular, the biological markers are markers associated with the signal transduction pathway of A3AR, i.e., elements that are known or that are experimentally found to be associated with the signal transduction mediated by A3AR. Examples are elements of the Wnt signal transudation pathway including: PKA, PKB, GSK-3β, β-catenin, cyclin D1 and c-myc; and elements of the NF-κB signal transudation pathway, such as PI3K, IKK, IKB, NF-κB and TNF-α.

The term “the level of a physiological parameter” refers to one of the following:

(1) the level of the biological marker's expression as determined by the amount of the biological marker's protein or protein fragment, or as determined by the amount of the biological marker's mRNA (which parameter is relevant to all markers, namely, markers associated with the Wnt and/or NF-κB pathways, such as A3AR, PKA, PKB, GSK-3β, NF-κB, IKK, PI3K, IKK, cyclin D1, β-catenin, c-myc, TNF-α and other);

(2) the phosphorylation level of the biological marker (which parameter is relevant to PKB, PKA and especially to GSK-3β and β-catenin); or

(3) the cellular localization of the biological marker, for example, localization in cell membrane vs. cytosolic (for A3AR), cytosol vs. nucleus (β-catenin, NF-κB).

Detecting the level of expression of the biological marker is carried out by any technique known in the art to detect the presence of a protein or a fragment of a protein in cells either at the cytosol or the membrane as well as by techniques for the detection of mRNA using any technique known in the art to detect the presence of a protein, or a fragment of a protein in cells either at the cytosol, at the membrane, or in any intracellular component of the cells, as well as in techniques for the detection of mRNA level in any component of the cells.

Methods for detecting the level of the protein may include: extracting the protein contents of the cells, or extracting fragments of protein from the membranes of the cells, or from the cytosol, for example, by using state of the art lysis, digestive, separation, fractionation and purification techniques, and separating the proteinaceous contents of the cells (either the crude contents or the purified contents) on a western Blot, and then detecting the presence of the protein, or protein fragment by various identification techniques known in the art. For example, the contents separated on a gel may be identified by using suitable molecular weight markers together with a protein identification technique, or using suitable detecting moieties (such as labeled antibodies, labeled lectins, labeled binding agents (agonists, antagonists, substrates, co-factors, ATP, etc.). The detection may also be by in situ, i.e., in the full tissue sample, by binding of specific recognition agents, to the biological markers when present in intact cells or in tissue, (relevant in connection with the present invention especially to determination of the level of A3AR). Such specific recognition agents may be labeled A3AR agonists, such as labeled IB-MECA; labeled A3AR antagonists such as labeled MRS1523, antibodies against A3AR etc. The presence of the labeled recognition moieties may be detected using techniques suited for the nature of the label. Where the recognition agents are fluorescent-labeled, the detection may be carried out by using a confocal microscope and directly viewing the level of the of the label bound (to the membranes). Where the recognition agents are labeled, for example, radio-labeled, the level may be determined by the determination of the radio-label level in the cells.

The determination of expression level may also be determination of mRNA level, for example, the detection may be by any methods used in the art for the detection of RNA in a cell-containing sample such as by using in situ hybridization with a detectable probe, for example, with a complementary sequence containing a detectable moiety (fluorescent, radioactive, chromatophoric moiety, etc). In such a case of in situ hybridization there is no need to extract the RNA from the cells and all that is needed is treatment to render the cells porous. However various amplification methods, which are sensitive enough to detect to minute amounts of RNA are preferable. Such methods include, PCR, RT-PCR, in situ PCR, in situ RT-PCR (all the above referring also to “nested” PCR, and nested RT-PCR), LCR (ligase chain reaction) and 3SR (self-sustained sequence replication). In accordance with a preferred embodiment RT-PCR and nested RT-PCR are used. The amplification products are identified by methods used in the art such as by separation on a gel and detection using a suitable labeled probe.

The sample may be membranes of tissue samples for example obtained by biopsy, intact cells separated from the tissue sample, or intact cells present in the circulation such as in the blood or any other body fluid, cells or tissue samples obtained from the subject including paraffin embedded tissue samples, proteins extracted obtained from the cytosol, cell membrane, nucleus or any other cellular component or mRNA obtained from the nucleus or cytosol.

Where the physiological parameter is for example the phosphorylation level of the marker, this may be determined by using labeled antibodies against phosphorylated substances such as labeled anti-tyrosine antibodies or antibodies that are capable of binding to phosphorylated GSK-3β.

Where the level of the tested parameter is localization in various cellular components, the amount of the marker in each compartment, or ratio of the amounts in various components may be determined. This may be done by separating the cellular components (for example lysing the cell and obtaining separately the membrane and the cytosol) or obtaining separately the cytosol and the nucleus and determining the protein content of the relevant biological marker in each separated cellular components, by using any one of the methods mentioned above or other methods used to determine protein contents.

Alternatively it is possible for determining A3AR localized, to use labeled A3AR binding agents (antibodies, agonists, antagonists) especially fluorescent labeled binding agents, and monitor the localization of the A3AR on the surface of the cells, for example using a confocal microscope.

In general, the physiological parameter may change in one of two manners as compared to control: a change indicative of increased proliferation (hereinafter “pro-proliferative”) as a result of treatment such as administration of a drug (preferably an A3AR modulator, most preferably an A3AR agonist), or a change indicative of decreased proliferation (hereinafter: “anti-proliferative”) as a result of treatment, such as administration of the drug.

Examples of changes indicative of anti-proliferative effect of the treatment:

(1) In the Expression Level: decrease in protein or mRNA expression of A3AR, PKB/Akt, PKA, β-catenin, c-myc, cyclin D1, NF-κB or TNF-α or increase in the protein or mRNA level of GSK-3β.

(2) In Phosphorylation Level: decrease in phosphorylation level of GSK-3β, increase in the phosphorylation level of PKB/Akt and PKA, and of β-catenin.

(3) In Localization: decrease in the localization of A3AR receptor in the cellular membrane as compared to control, decrease in the localization of β-catenin in the nucleus as compared to cytosol; decrease in the localization of NF-κB in the nucleus as compared to the cytosol.

Changes indicative of pro-proliferative effects of the treatment include:

(1) In the Expression Level: increase in protein or mRNA expression of A3AR, PKB/Akt, PKA, β-catenin, c-myc, cyclin D1 and NF-κB, or decrease in the protein or mRNA expression level of GSK-3β.

(2) In Phosphorylation Level: increase in phosphorylation level of GSK-3β decrease in the phosphorylation level of PKB/Akt and PKA and of β-catenin.

(3) In Localization: increase in the localization of A3AR receptor in the cellular membrane as compared to control, increase in the amount of β-catenin and NF-κB in the nucleus as compared to cytosol.

The changes indicative of a decreased proliferation show effectiveness of an A3AR modulator, preferably an A3AR agonist, administered for the treatment of a disease state wherein a therapeutically beneficial effect may be evident by decrease or inhibition of proliferation. Examples of such diseases that are typically characterized by excess proliferation include, without being limited thereto, all types of cancer; and, in particular, all types of solid tumors; skin proliferative diseases (e.g., psoriasis); a variety of benign hyperplastic disorders; inflammatory diseases; and others.

The term “solid tumors” refers to carcinomas, sarcomas, adenomas, and cancers of neuronal origin and, in fact, to any type of cancer which does not originate from the hematopoietic cells and in particular concerns: carcinoma, sarcoma, adenoma, hepatocellular carcinoma, hepatocellular carcinoma, hepatoblastoma, rhabdomyosarcoma, esophageal carcinoma, thyroid carcinoma, ganglio blastoma, fibrosarcoma, myxosarcoma, liposarcoma, chondrosarcoma, osteogenic sarcoma, chordoma, angiosarcoma, endotheliosarcoma, lymphangiosarcoma, synovioma, Ewing's tumor, leiomyosarcoma, rhabdotheliosarcoma, colon carcinoma, pancreatic cancer, breast cancer, ovarian cancer, prostate cancer, squamous cell carcinoma, basal cell carcinoma, adenocarcinoma, renal cell carcinoma, hematoma, bile duct carcinoma, melanoma, choriocarcinoma, seminoma, embryonal carcinoma, Wilms' tumor, cervical cancer, testicular tumor, lung carcinoma, small lung carcinoma, bladder carcinoma, epithelial carcinoma, glioma, astrocytoma, medulloblastoma, craniopharyngioma, ependymoma, pinealoma, retinoblastoma, multiple myeloma, rectal carcinoma, thyroid cancer, head and neck cancer, brain cancer, cancer of the peripheral nervous system, cancer of the central nervous system, neuroblastoma, cancer of the endometrium, as well as metastasis of all the above. It has been shown in accordance with the invention that increased expression of A3AR can be found not only in the primary tumor site but also in metastasis thereof.

Benign hyperplastic disorders include, without being limited thereto, benign prostate hyperplasia (BPH), non-tumorigenic polyps in the digestive tract, in the uterus and others.

Inflammatory diseases include, without being limited thereto, rheumatoid arthritis, Crohn's disease, multiple sclerosis and others. It has been found in accordance with the invention that inflammatory cells at the site that exhibits the inflammatory cells and at times also in draining lymph nodes, that increase A3AR expression and consequently characteristic changes of the physiological parameters is found in such cells.

Where the disease is cancer the cells that are obtained from the subject may be cells suspected of being transformed as well as other cells notably blood cells such as neutrophils. Cells suspected of being transformed may be obtained by methods known for obtaining “suspicious” cells such as by biopsy, needle biopsy, fine needle aspiration and others; the suspicion of being in a disease state, may be incurred due to various imaging (NMR, MR, scanning, ultrasound, mammography) or pathological techniques. Blood cells may be obtained simply by drawing blood.

The changes indicative of an increased proliferation show effectiveness of a drug (preferably an A3AR modulator, most preferably an A3AR agonist) administered for the treatment of a disease or a condition wherein a therapeutically beneficial effect may be evident by increase proliferation. These conditions are typically conditions where normal cells die as a result of trauma (injury, ischemia, hypoxia) or as a result of administration of a toxic substances, or as a result of toxic treatment such as substances or radiation administered during the course of chemo- or radio-therapy. In particular this term refers to the effect of myelo-protective therapy, i.e., prevention of a decrease in the number of neutrophils, and other white blood cells following chemo- or radio-therapy.

The agent that interacts with the A3AR is an A3AR agonist or antagonist and, in accordance with a preferred embodiment, is an A3AR agonist.

The term “A3AR-associated signal transudation pathway” concerns any pathways that begins by activation by the A3AR receptor and continues by the effect of its downstream effectors. These effectors are currently known to include elements of the Wnt pathway and the NF-κB pathway, but further elements are constantly being discovered and this term covers such newly found elements as well as newly found pathways associated with A3AR activation.

The sample assayed in accordance with the invention refers to cells, tissue samples or cell components (such as cellular membranes or cellular components) obtained from the treated subject. By one embodiment the sample are cells known to manifest the disease, for example, where the disease is cancer of type X, the cells are the cells of the tissue of the cancer (breast, colon, skin, liver, lungs, cells, etc.) or metastasis of the above. By another embodiment the sample may be non-diseased cells such as cells obtained from blood for example neutrophils.

The “diseased state” wherein effectiveness is indicated by decreased proliferation, includes tumors, and in particular solid tumors, examples of solid tumors being melanoma, colon carcinoma, prostate carcinoma, lung cancer, breast cancer, pancreatic cancer, and skin proliferative diseases, such as psoriasis.

The diseased state wherein the effectiveness is indicated by increased proliferation, are in particular diseases wherein there is an increase in the blood count of white cells, such as neutrophils, as a result of chemo- or radio-therapy.

Monitoring the levels of at least one physiological parameter of the biological marker in the cells, in accordance with one of the uses of the above method, may also help to screen for likely candidates for treatment of a diseases state through interaction with the A3AR. Particular examples are A3AR agonists for treatment of cancer or inflammatory diseases.

For example, it is possible to use cell cultures of the diseased state, for example, a specific line of cancer cells derived from a type of cancer that is the intended target for therapy. Determination of modulation of one or more of said physiological parameters may serve as an indication for possible use of the drug candidate in treating the cancer.

In accordance with the invention, it has been found that there are drug-induced fluctuations in the level of expression or in the cellular localization of various biological markers after the administration of the A3AR modulator and, in particular, A3AR agonist, that occur following drug administration and exposure of the cells thereto. This means that if, for example, the amount of the protein of a specific marker decreased as a result of A3AR-modulator administration, this decrease may be most prominent X minutes after administration of the A3AR modulator, while after 2X minutes the decrease may be less prominent and after 3X minutes the decrease may be prominent again. This fluctuative behavior (typically in a sinusoidal-like pattern) characterizes the protein level of most biological markers as well as the localization on the cellular membrane of the A3AR receptors itself. It is clear that it is best to carry out the method of the invention at a certain time period after administration of the drug wherein the change in the level of the parameter is most prominent as compared to control.

Thus, before carrying out the method of the invention it may be important to carry out a preliminary determination to establish the optimal time after the drug administration wherein the changes in the physiological parameters are most prominent as compared to control. This may be achieved by administering the drug and monitoring the fluctuations in the physiological parameters (in vitro, in vivo, but most preferably in a number of subjects wherein the sample is obtained at a number of different time periods after administration, and choosing as the time for determination the period wherein the difference between the parameters in the treated sample (cells, tissue, etc) is the greatest as compared to the untreated control.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIGS. 1a-1c shows confocal laser microscopy analysis of B16-F10 melanoma cells labeled with the primary and secondary antibodies against A3AR and the Cy3-conjugated anti-goat IgG, respectively; wherein FIG. 1a exhibits an image of A3AR in untreated melanoma cells; FIG. 1b exhibits IB-MECA treated cells; and FIG. 1c exhibits a combined treatment with IB-MECA and MRS an A3AR antagonist. Images represent the center section of the X-Y plane.

FIG. 2 shows confocal laser microscopy analysis of B16-F10 melanoma cells labeled with the primary antibody against A3AR and the secondary antibody, Cy3-conjugated anti-goat IgG, respectively, exposed for different time periods to IB-MECA. Images represent the center section of the X-Y plane.

FIG. 3 shows Western blot analysis of A3AR internalization in untreated melanoma cells (control), or upon trypsin treatment, IB-MECA treatment, or a combined treatment with IB-MECA and trypsin. The level of the housekeeping protein β-actin did not change.

FIGS. 4a-4b show Western blot analysis of A3AR in B16-F10 melanoma cells; wherein FIG. 4a presents results upon treatment of the cells for different time periods with IB-MECA; and FIG. 4b presents results upon treatment with IB-MECA, a combination of IB-MECA and cycloheximide (protein synthesis inhibitor) or a combination of IB-MECA with MG132 (protein degradation inhibitor), as compared to untreated cells (control).

FIGS. 5a-5b show the correlation between tumor size and the level of regulatory elements in colon carcinoma cells, wherein FIG. 5a presents tumor size after 15 days of daily treatment with IB-MECA of mice inoculated with B16 melanoma cells, while FIG. 5b presents the modulation of cell growth regulatory proteins (PKAc, PKB/Akt, GSK-3β, β-catenin cyclin D1, c-myc and NF-κB) in the tumor lesions described in FIG. 5a (left prior to treatment with A3AR agonist; right after treatment with A3AR agonist).

FIG. 6 shows immunoblot analysis of proteins extracted from prostate carcinoma cells, being NF-κB, c-myc and cyclin D1 is in the presence (right lane) and absence (left lane) of IB-MECA.

FIG. 7 shows immunoblot analysis of protein extracts derived from colon carcinoma cell, being PKAc, PKB/Akt, β-catenin, c-myc and cyclin D1 NF-κB and GSK-3β in the presence (right lane) and absence (left lane) of IB-MECA.

FIGS. 8a-8c show Western blot analysis of receptor functionality in B16-F10 melanoma cells determined by monitoring the level of PKA and GSK-3β; wherein FIG. 8A presents the effect of IB-MECA on PKA and GSK-3β levels in the melanoma cells, at different time periods; FIG. 8B presents the effect on cells treated simultaneously with IB-MECA+MRS 1523, and FIG. 8C presents the effect on cells treated with Forskolin or 8-Br-cAMP.

FIGS. 9a-9b show the correlation between tumor size and the level of regulatory elements in colon carcinoma cells, wherein FIG. 9a presents tumor size after 15 days of daily treatment with IB-MECA of mice inoculated with HCT-116 human colon carcinoma as a function of time; while FIG. 9b presents the modulation of cell growth regulatory proteins (A3AR, GSK-3β, cyclin D1 and c-myc) in the tumor lesions described in FIG. 9a.

FIG. 10 shows detection of A3AR receptor on human neutrophils;

FIGS. 11a-11b show the anti-inflammatory response of IB-MECA. FIG. 11A shows the clinical score of 10 μg/Kg of IB-MECA (▪) as compared to vehicle-treated control (▴) and the nullification of the anti-inflammatory response by the specific A₃ receptor antagonist MRS 1220 administered 30 minutes prior to the administration of IB-MECA (♦). FIG. 11B shows the histological score of the IB-MECA treated rats of FIG. 11A at day 28 as compared to control.

FIG. 12 is a Western blot showing the modulation of A3AR and downstream signaling effector PI3K in drain lymph nodes (DLN) in an adjuvant induced arthritis (AIA) rat model. Shown are blots of A3AR, of A₂ adenosine receptor (A2AR) serving as a negative control demonstrating the specificity of the A3AR modulation, and blots of PI3K. The left blot is from a control, vehicle treated animal; the center from an IB-MECA treated animal and the right one from an animal treated with both IB-MECA and MRS 1220, which is a specific A3AR antagonist.

FIGS. 13a-b demonstrate the IB-MECA-induced modulation in the level of PKB/Akt in DLN in an AIA rat model, shown both in terms of protein expression (FIG. 13A) and in terms of activity utilizing GSK-3β as a substrate (FIG. 13B). FIG. 13A is a Western blot of phosphorylated PKB/Akt. FIG. 13B is a Western blot of phosphorylated GSK-3β. The left blot in each case is from a control, vehicle treated animal; the center from an IB-MECA treated animal and the right one from an animal treated with both IB-MECA and MRS 1220. The β-actin blots in FIG. 13A are given as control to demonstrate the specificity of the response.

FIG. 14 is a Western blot demonstrating the IB-MECA induced modulation of IKKα/β, NF-κB and TNF-α in DLN of an AIA rat model. The NF-κB level is shown by a DNA binding assay.

FIG. 15 is a Western blot demonstrating the IB-MECA induced modulation of IKKα/β, NF-κB and TNF-α in synovial tissue from an AIA rat model.

FIG. 16 shows the IB-MECA induced up-regulation of GSK-3β and Caspace-3 in DLN of AIA rat model.

### DETAILED DESCRIPTION OF THE INVENTION

The present invention is based on the finding that there exists a cross-talk between A3AR and the Wnt and NF-κB signaling pathways. A3AR activation was found in cancer cells, to inhibit PKA and PKB/Akt thereby retaining GSK-3β in its active non-phosphorylated form (Fishman et al, 2002). Active (non-phosphorylated) GSK-3β was shown to phosphorylate and inactivate β-catenin, eliminating its migration to the nucleus and consequently inducing the down-regulation of c-myc and cyclin D1 (Ferkey et al, 2000) resulting in decreased proliferation of the cells.

In the NF-κB pathway the decrease in PKB/Akt level leads to down-regulation of IKK and NF-κB which will prevent the release of the latter from its complex with IKB and its entry to the nucleus, thus preventing the translocation of NF-κB to the nucleus, resulting in a decreased induction the transcription of cyclin D1 and c-myc, leading to a decreased in proliferation. The NF-κB is also the transcription factor of TNF-α and thus the down-regulation of NF-κB also leads to down-regulation of this cytokine.

In proliferative diseases such as in tumors, including, for example, melanoma, colon carcinoma, prostate carcinoma as well as in other tumor cells, the untreated course of events, is manifested by changes in the physiological parameters of the biological markers in the pro-proliferative direction including failure of the phosphorylated GSK-3β to phosphorylate β-catenin, which thus accumulates in the cytosol. It then translocates to the nucleus where it induces the transcription of cyclin D1 and c-myc, leading to cell cycle progression (Bonvini et al, 2000; Olah et al, 2000). Successful anti-proliferative treatment, monitored by the determination of the following changes in the level of the physiological parameters of the biological markers is a change of the physiological parameters of the biological markers in the anti-proliferative direction.

In some autoimmune inflammatory diseases, activation of the A3AR with an agonist to this receptor induces inhibition of PI3K and PKB/Akt leading to apoptosis and inhibition of production of TNF-α. Thus monitoring the level of these three proteins may provide a means to gauge the anti-inflammatory effect of and A3AR agonist.

The following are changes in the physiological parameters that mark successful treatment of disease wherein a beneficial effect is evident by decreased proliferation such as cancer:

(1) In the Expression Level: decrease in protein or mRNA expression of A3AR, PKB/Akt, PKA, β-catenin, c-myc, cyclin D1, PI3K, IKK, NF-κB or TNF-α, or increase in the protein or mRNA level of GSK-3β.

(2) In Phosphorylation Level: decrease in phosphorylation level of GSK-3β, increase the phosphorylation level of PKB/Akt and PKA and β-catenin.

(3) In Localization: decrease in the localization of A3AR receptor in the cellular membrane as compared to control, decrease in the localization of β-catenin and NF-κB, in the nucleus as compared to cytosol.

The following are changes in the physiological parameters of the biological markers that are indicators of successful treatment of diseases and conditions wherein a therapeutically beneficial effect may be evident by the increase of proliferation such as increase in the proliferation of white blood cells following chemo- or radio-therapy:

(1) In the Expression Level: increase in protein or mRNA expression of A3AR, PKB/Akt, PKA, β-catenin, c-myc, cyclin D1 and NF-κB, or decrease in the protein or mRNA expression level of GSK-3β.

(2) In Phosphorylation Level: increase in phosphorylation level of GSK-3β, decrease in the phosphorylation level of PKB/Akt and PKA and β-catenin.

(3) In Localization: increase in the localization of A3AR receptor in the cellular membrane as compared to control, increase in the amount of β-catenin and NF-κB in the nucleus as compared to cytosol.

### SPECIFIC EXAMPLES

A. Materials and Methods

Rabbit polyclonal antibodies against murine and human A3AR, PKAc, c-myc and GSK-3β were purchased from Santa Cruz Biotechnology Inc., Ca, USA.

Rabbit polyclonal antibodies against murine and human cyclin D1 (Upstate, N.Y.), A2B adenosine receptor, Cy3-conjugated anti-goat IgG and Fluorescein-conjugated anti-rabbit IgG were purchased from Chemicon, Ca.

Cycloheximide and Forskolin were obtained from Sigma, St Louis, and 8-Br cAMP and MG132 from Calbiochem, Ca.

Male ICR mice aged 2 months, weighing an average of 25 g as well as Nude/BalbC male, 10 weeks old mice (Harlan Laboratories, Jerusalem, Israel) and rats (Harlan Laboratories, Jerusalem, Israel), were employed in the following experiments. Standardized pelleted diet and tap water were supplied.

Experiments were performed in accordance with the guidelines established by the Institutional Animal Care and Use Committee at the Rabin Medical Center, Petach Tikva, Israel.

B. Immunostaining and Confocal Microscopy

B16-F10 melanoma cells were grown for 24 hours on cover slips coated with Ploy-L-Lysine (500 μg/ml). Cells were fixed in 4% formaldehyde in phosphate-buffered saline (PBS) for 1 hour at room temperature. The fixed cells were rinsed three times for one min with PBS. To block nonspecific interaction of the antibodies, cells were incubated for 30 minutes in 4% normal goat serum (NGS) in PBS (1% bovine serum albumin (BSA), 0.1% Triton X-100). For the single labeling experiments, cells were then incubated with the primary antibodies against A3AR at a dilution of 1:1000 in PBS (1% BSA, 1% NGS, 0.1% Triton X-100) for 24 hours at 4° C. For the double labeling experiments, antibodies against the A_{2B} adenosine receptor (A₂BR) were added at a dilution of 1:1000 to the reaction mixture. After being washed three times for 3 minutes with PBS, cells were incubated with Cy3-conjugated anti-goat IgG for the single labeling experiment, and with both, Fluorescein-conjugated anti-rabbit IgG and Cy3 for the double labeling. Both antibodies were diluted to 1:250 in PBS and incubated in the dark for 2 hours. Cells were rinsed with PBS three more times and mounted with AM 100 (Chemicon, Ca). Stained cells were visualized by Confocal Microscope (Zeiss, Axiovert 100 M, excitation at 553 and emission at 560-615 nm for Cy3, and 492 and 520 nm, respectively, for Fluorescein).

C. Western Blot Analysis

To detect the level of expression of the A3AR proteins Western blot analysis was performed. Cells were incubated in the presence and absence of IB-MECA (10 nM), MRS1523 (10 nM), Forskolin (50 nM), 8 Br cAMP (100 μM), Cycloheximide 20 μg/ml or MG 132 (20 nM) for different time periods at 37° C. Cells were then rinsed with ice-cold PBS and transferred to ice-cold lysis buffer (TNN buffer, 50 mM Tris buffer pH=7.5, 150 mM NaCl, NP 40). In the experiment examining the effect of trypsin on A3AR expression, cells were incubated with 0.5 ml of 0.25% trypsin for 5 minutes. The trypsinized cells were washed again with ice-cold PBS, harvested by centrifugation and subjected to lysis in TNN buffer. Cell debris was removed by centrifugation for 10 minutes, at 7500 xg. The supernatant was utilized for Western Blot analysis. Protein concentrations were determined using the Bio-Rad protein assay dye reagent. Equal amounts of the sample (50 μg) were separated by SDS-PAGE, using 12% polyacrylamide gels. The resolved proteins were then electroblotted onto nitrocellulose membranes (Schleicher & Schuell, Keene, NH, USA). Membranes were blocked with 1% bovine serum albumin and incubated with the desired primary antibody (dilution 1:1000) for 24 hours at 4° C. Blots were then washed and incubated with a secondary antibody for 1 hour at room temperature. Bands were recorded using BCIP/NBT color development kit (Promega, Madison, W1, USA). Data presented in the different figures are representative of at least three different experiments.

D. Northern Blot Analysis

Total RNA was isolated from B16-F10 melanoma cells treated with IB-MECA, utilizing Tri-reagent (Sigma, Saint Louis). The samples were then subjected twice to phenol:chloroform extraction and washed with chloroform. RNA was precipitated with sodium acetate/ethanol following washing with ethanol, then denatured, separated (25 μg per lane) in 1.1% formaldehyde agarose gels and transferred to Hybond-N membrane. The 390 bp EcoRI fragment from A3AR cDNA clone of mouse (TAA3I.S), kindly supplied by Dr Kathia Ravid, was prepared by random-primed synthesis. Probes were used in RNA blot analysis at a hybridization temperature of 42° C. in the presence of 50% formamide.

E. RT-PCR-for Detection of Intact RNA in Formalin-Fixed Paraffin-Embedded Tissues

Tissue sections (5 μm thick) on slides that, stained by H&E were observed by a pathologist. The neoplastic area and the normal area were detected and each one marked separately. The neoplastic tissue and the normal tissue were collected to different microcentrifuge tubes. The samples were treated with proteinase K at a final concentration of 0.1 mg/ml and incubated at 37° C. for 1 hour to allow for DNA digestion. Cells lysate were heated to 95° C. for 15 minutes in order to inactivate DNase and proteinase K. Following centrifugation at 14,000 RPM for 5 minutes, 17 μl of the supernatant was transferred to separate tube and 4 μl of RT mixture. 5 mM dNTPs, 2.5 μM random hexamer, 5 U RNasin, 100 U SuperScript One Step RT-PCR with Platinum Taq (Invitrogene) were added.

The RT reaction was performed at 45° C. for 45 minutes. The PCR reaction was changed dependent on the primers used for the amplification. For set No I, the RT was followed by heating to 99° C. for 5 minutes, 50 cycles of 94° C. for 30 seconds, 59° C. for 45 seconds and 73° C. for 45 seconds were performed. For set No II, the annealing was done at 55° C. Products were electrophoresed on 2% agarose gels, stained with ethidium bromide and visualized with UV illumination. The specificity of the RT-PCR reaction was confirmed by size determination on agarose gels in comparison to a positive control, from RNA extracted using standard techniques and by sequencing the RT-PCR product and comparing the sequences to the known sequences (ADORA3-L77729, L77730).

TABLE 1
Nucleotide Sequence of A₃ Primers

| | | |
|---|---|---|
| | Fragment | |
| Gene | Size | Primer Sequence |

| | | | | |
|---|---|---|---|---|
| A3AR | 153 | (Set No I) | 5′-ACGGTGAGGTACCACAGCTTGTG | (SEQ ID NO:1) |
| | | | 3′-ATACCGCGGGATGGCAGACC | (SEQ ID NO:2) |
| | 361 | (Set No II) | 5′-ACCCCCATGTTTGGCTG | (SEQ ID NO:3) |
| | | | 3′-GCACAAGCTGTGGTACCTCA | (SEQ ID NO:4) |

F. In Vivo Studies

Mice were maintained on a standardized pelleted diet and supplied with tap water. Experiments were performed in accordance with the guidelines established by the Institutional Animal Care and Use Committee at the Rabin Medical Center, Petach Tikva, Israel.

Melanoma: C57BL/6J, male mice (Harlan Laboratories, Jerusalem, Israel) aged 2 months, weighing an average of 25 g were used. B16-F10 (2.5×105) melanoma cells were subcutaneously (s.c.) injected to the flank of the mice. IB-MECA at a dose of 100 μg/kg body weight was administered orally once daily, starting 24 hours after tumor cells' inoculation. The control group was treated daily, orally, with the vehicle only. Mice were sacrificed after 15 days, tumor lesions were excise and protein was extracted for measurements as described above. The tumor size (width (W) and length (L)) was measured with a caliber and calculated according to the following formula: Tumor Size =(W)2xL/2. Each group contained 15 mice and the study was repeated 3 times.

Colon Carcinoma: Nude/BalbC male, 10-week-old mice were used. HCT-116 Human colon carcinoma cells (1.2×10⁶ cells in 100 μL PBS) were inoculated subcutaneously to the flank of the mice. Treatment was initiated when tumors reached a size of ˜150 mm³. Each group contained 10 mice, the control group treated with vehicle only, while the test group was administered daily orally with IB-MECA (10 μg/kg).

After 32 days the mice were sacrificed, tumor lesions were excise and protein was extracted for measurements as described above. Tumor size was evaluated by measuring with a caliber width (W) and length (L) and calculated according to the above formula.

AIA in Rats: Female Lewis rats, aged 8-12 weeks, obtained from Harlan Laboratories (Jerusalem, Israel), were injected subcutaneously (s.c.) at the tail base with 100 μl of suspension composed of incomplete Freund's adjuvant (IFA) with 10 mg/ml heat killed Mycobacterium tuberculosis, (Mt) H37Ra, (Difco, Detroit, USA). To assess the clinical disease activity score, the animals were inspected every second day for clinical arthritis. The scoring system ranged from 0-4 of each limb: 0-no arthritis; 1-redness or swelling of one toe/finger joint; 2-redness and swelling of more than one toe/finger joints; 3-the ankle and tarsal-metatarsal joints involvement; 4-entire paw redness or swelling. The arthritic score was calculated by adding the four individual legs' score to a maximum of 16. For histological scoring, Animals were sacrificed, the legs were removed up to the knee level, fixed in 10% formaldehyde, decalcified, dehydrated, paraffin-embedded, cut into 4 μm sections and stained by Hematoxylin-Eosin. The assessment of all pathologic findings were performed blind using semi-quantitative grading scales of 0 to 4 for the following parameters: a) extent of inflammatory cells' infiltration to the joint tissues; b) synovial lining cell hyperplasia; c) pannus formation; and d) joint cartilage layers destruction. A fifth parameter, bone damage and erosion, was graded 0-5: 0-normal; 1-minimal loss of cortical bone at a few sites; 2-mild loss of cortical trabecular bone; 3-moderate loss of bone at many sites; 4-marked loss of bone at many sites; 5-marked loss of bone at many sites with fragmenting and full thickness penetration of inflammatory process or pannus into the cortical bone The mean of all the histological parameters scores were designated “Histology Score”. TNF-α was measured in synovia, DLN and spleen tissues derived from control and CF101 treated rats. To detect the level of expression of TNF-α, WB analysis was performed. Spleen and DLN mononuclear cells and synovial tissue were rinsed with ice-cold PBS and transferred to ice-cold lysis buffer (TNN buffer, 50 mM Tris buffer pH=7.5, 150 mM NaCl, NP 40 0.5% for 20 minutes). Cell debris was removed by centrifugation for 10 minutes, at 7500 g. The supernatant was utilized for WB analysis. Protein concentrations were determined using the Bio-Rad protein assay dye reagent. Equal amounts of the sample (50 μg) were separated by SDS-PAGE, using 12% polyacrylamide gels. The resolved proteins were then electro-blotted onto nitrocellulose membranes (Schleicher & Schuell, Keene, NH, USA). Membranes were blocked with 1% bovine serum albumin and incubated with polyclonal goat anti-rat TNF-α antibodies (dilution 1:1000) (Santa Cruz Biotechnology Inc., CA, USA) for 24 hours at 4° C. Blots were then washed and incubated with rabbit anti-goat polyclonal antibodies for 1 hour at room temperature. Bands were recorded using BCIP/NBT color development kit (Promega, Madison, Wis., USA). Densitometry of protein expression was normalized against β-actin and expressed as % of control.

G. Statistical Analysis

The results were evaluated using the Student's t-test, with statistical significance at p<0.05. Comparison between the mean value of different experiments was carried out.

### Example 1

Monitoring Changes in Biological Marker Localization in Melanoma Cells

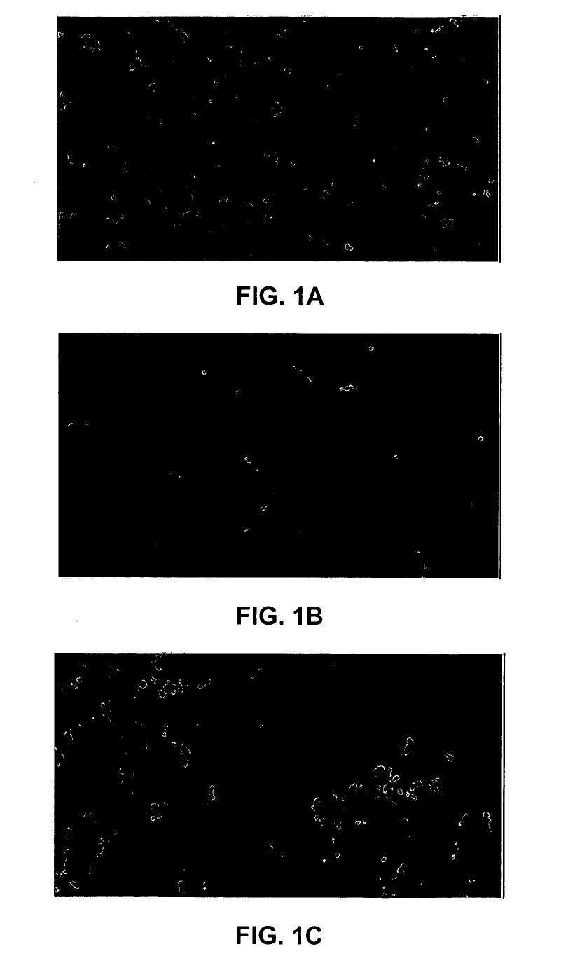

To study receptor localization, confocal laser microscopy was utilized. Untreated cells (control) highly exhibited A3AR on the cell surface, as seen from the fluorescence intensity level (FIG. 1a). A marked decrease in the fluorescence level was noted after 5 minutes in the IB-MECA treated cells (FIG. 1b). Exposure of the melanoma cells to the A3AR antagonist MRS1523, in the presence of IB-MECA, resulted in cell surface fluorescence intensity similar to that of the control (FIG. 1c). These data suggest that rapid receptor internalization took place upon IB-MECA treatment.

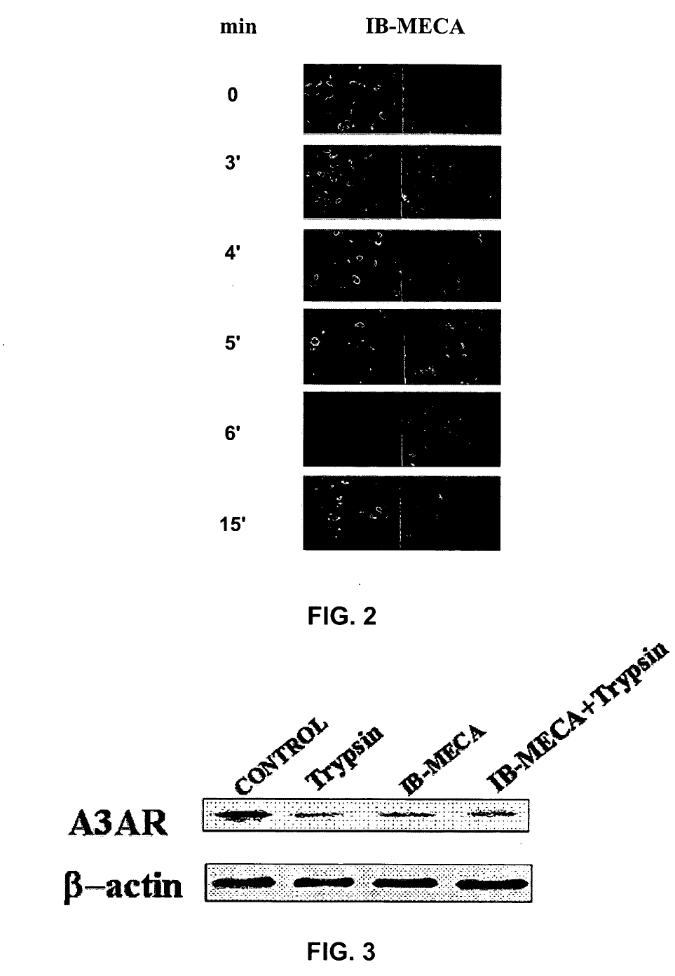

To further explore the time course kinetic of A3AR internalization, B16-F10 melanoma cells were exposed for different time periods to IB-MECA and confocal microscopy analysis was carried out. FIG. 2 depicts the gradual internalization rate that occurred within a few minutes, resulting in the disappearance of the fluorescence after 6 minutes. Prolonged exposure (15 minutes) of the melanoma cells to IB-MECA resulted in receptor externalization to the cell surface. This was followed by internalization/externalization after longer incubation time periods (30 and 60 minutes). To confirm the observation that the fluorescence level is decreased as a result of internalization, optical sectioning of the cells was performed (data not shown). In untreated cells (controls) the receptor was exhibited on the cell surface, and upon exposure to IB-MECA (for 5 minutes), it was presented in the cytoplasm, supporting the notion that A3AR translocates from the membrane to the cytosol. After 15 and 60 minutes' exposure, the receptor was seen both in the cytoplasm and on the cell surface. In the 60-minute samples, more fluorescence was seen in the cytosol in comparison to the 15-minute samples. To show the specificity of A3AR localization, it was compared to that of the A_{2B} adenosine receptor. Interestingly, in the untreated cells, while A3AR was mainly localized on the cell surface, the A_{2B}R was seen in the cytoplasm. Upon exposure of the cells to IB-MECA, the A_{2B}R was masked by A3AR in the cytoplasm.

### Example 2

Monitoring Changes in RNA and Protein Expression Level of A3AR in IB-MECA Administered Melanoma Cells

Receptor internalization by treating melanoma cells with trypsin, which removes the cell surface receptor, was examined. Untreated melanoma cells expressed a high level of A3AR protein, which was down-regulated after IB-MECA or trypsin treatment (FIG. 3. lanes 1, 2 and 3). This indicates that in the control untreated tumor cells most A3AR is exhibited on the cell surface and is subjected to digestion by trypsin. In the IB-MECA+trypsin treated cells (5 minutes), a negligible difference was observed between the trypsin treated and untreated cells, demonstrating that most A3AR was already internalized (change in localization) due to the treatment with the A3AR agonist IB-MECA, thus protected from digestion (FIG. 3a, lanes 3 and 4).

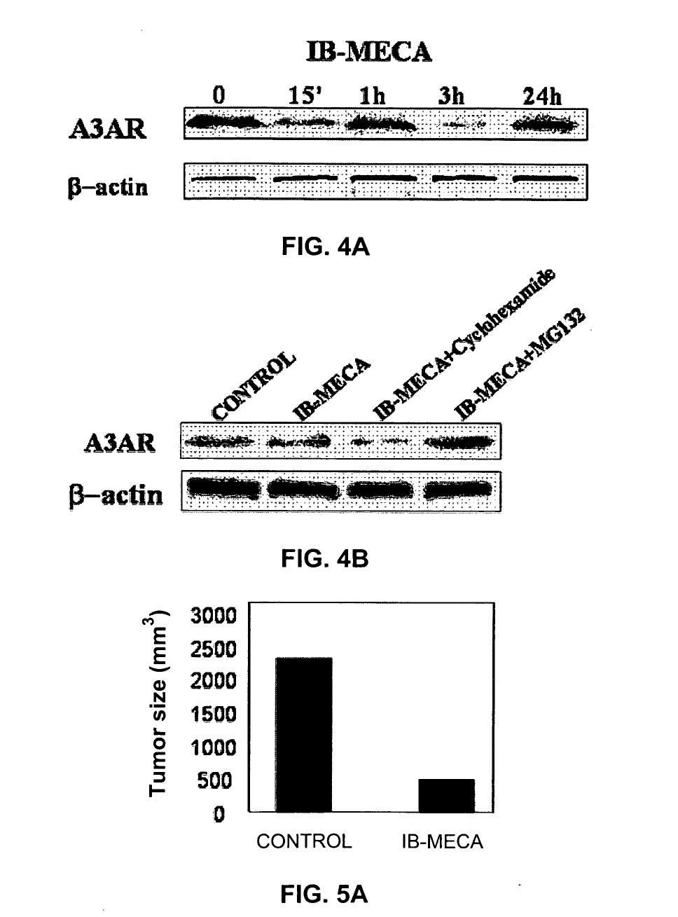

Time dependent expression of A3AR in the melanoma cells was examined by Western blot analysis. IB-MECA induced modulation of A3AR expression in a sinusoidal pattern, i.e., down-regulation and up-regulation, occurred at different time points (FIG. 4a.). This pattern indicted the optimal time for carrying out the method of the invention has to be determined experimentally.

To test whether protein expression was modulated due to degradation and re-synthesis, the cells were exposed for 180 minutes to IB-MECA in the presence of MG132 (protein degradation inhibitor) and cycloheximide (protein synthesis inhibitor). Indeed, MG132 prevented A3AR down-regulation and cycloheximide inhibited receptor up-regulation, illustrating that following internalization, receptor degradation and re-synthesis took place (FIG. 4b). Moreover an increase in mRNA expression level was observed, suggesting that a de novo synthesis of A3AR had occurred (FIG. 4c). The specificity of this response was demonstrated by MRS1523 which reversed the increase in mRNA expression

### Example 3

Monitoring Expression Level of Biological Markers in Melanoma Inoculated Mice Treated with IB-MECA

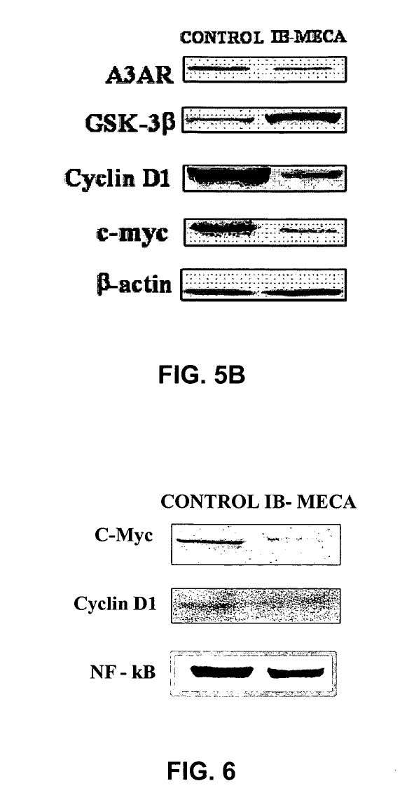

IB-MECA markedly suppressed the development of B16-F10 melanoma tumor growth (76% inhibition, p<0.0001, FIG. 5a). In tumor lesions excised from these mice, Western blot analysis (FIG. 5b) revealed that the levels of both PKA and PKB/Akt are decreased. Consequently, an increase in the total GSK-3β level was noted, thereby leading to a decrease in the level of β-catenin. Cyclin D1 and c-myc, known to be transcripted following translocation of β-catenin/Lef1 to the cell nucleus, were both found to be down-regulated in the IB-MECA treated melanoma cells.

In addition, the level of the transcription factor NF-κB, which its activation plays an important role in tumor development was also down-regulated. The level of the housekeeping protein β-actin did not change (not shown).

### Example 4

Monitoring Expression Level of Biological Markers During Inhibition of Colon Carcinoma Development in Mice by IB-MECA Treatment

IB-MECA markedly suppressed the development of colon carcinoma cells in mice inoculated with HCT-116 human colon carcinoma cells (FIG. 5a). In tumor lesions excised from mice inoculated with the HCT-116 human colon carcinoma cell, Western blot analysis revealed down-regulation of A3AR, up-regulation of GSK-3β expression level, followed by a decrease in the level of c-myc and cyclin D1 (FIG. 5b). The level of the housekeeping protein β-actin did not change

### Example 5

Monitoring Change of Expression of Biological Markers in Prostate Carcinoma (PC-3)

The effect of IB-MECA on key proteins downstream to A3AR activation in prostate carcinoma cells was also examined in a manner similar to that performed with melanoma cells (see above). FIG. 6 presents immunoblot analysis of proteins extracted from prostate carcinoma cells, wherein down-regulation of A3AR, NF-κB, c-myc and Cyclin D1 is exhibited.

### Example 6

Monitoring Changes of Expression Level of Biological Markers in Colon Carcinoma Cells as a Result of IB-MECA Administration

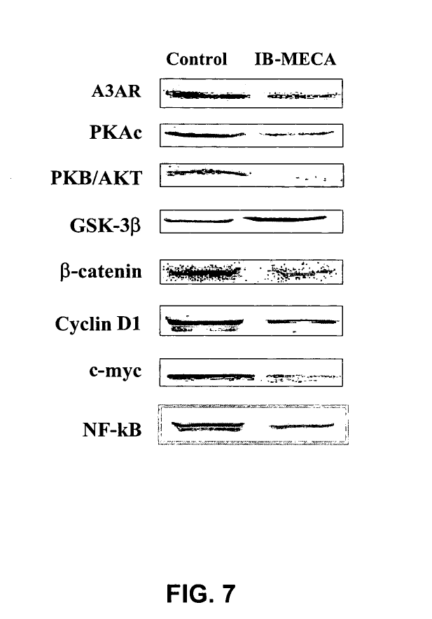

The effect of IB-MECA on key proteins downstream to A3AR activation in HCT-116 human colon carcinoma cells was also examined in a manner similar to that performed with melanoma cells (see above). FIG. 7 presents Immunoblot analysis of protein extracts derived from colon carcinoma cell, wherein treatment with IB-MECA (right lane) caused down-regulation of PKAc, PKB/Akt, β-catenin, c-myc and cyclin D1 and NF-κB and up-regulation of GSK-3β expression level as compared to control (left lane). These results conform with the results obtained with melanoma, and prostate cancer cells and presented hereinabove and support the notion that determination of the expression level of these regulatory elements may function as biological markers for disease states.

It should be noted that the level of the transcription factor NF-κB, which its activation plays an important role in tumor development was also down-regulated.

### Example 7

Monitoring Expression of Key Proteins Downstream to A3AR Activation by IB-MECA Treatment in Melanoma Cells

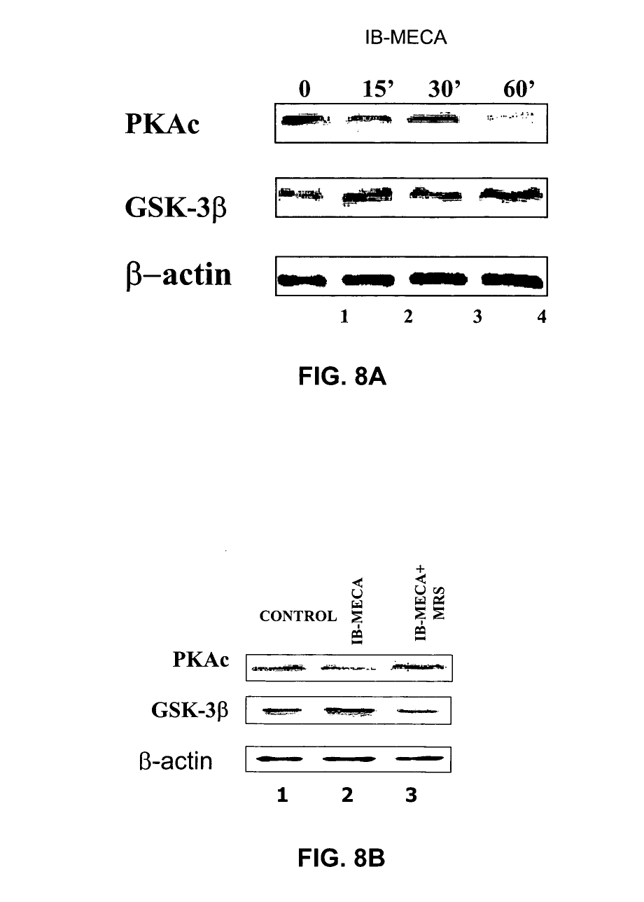

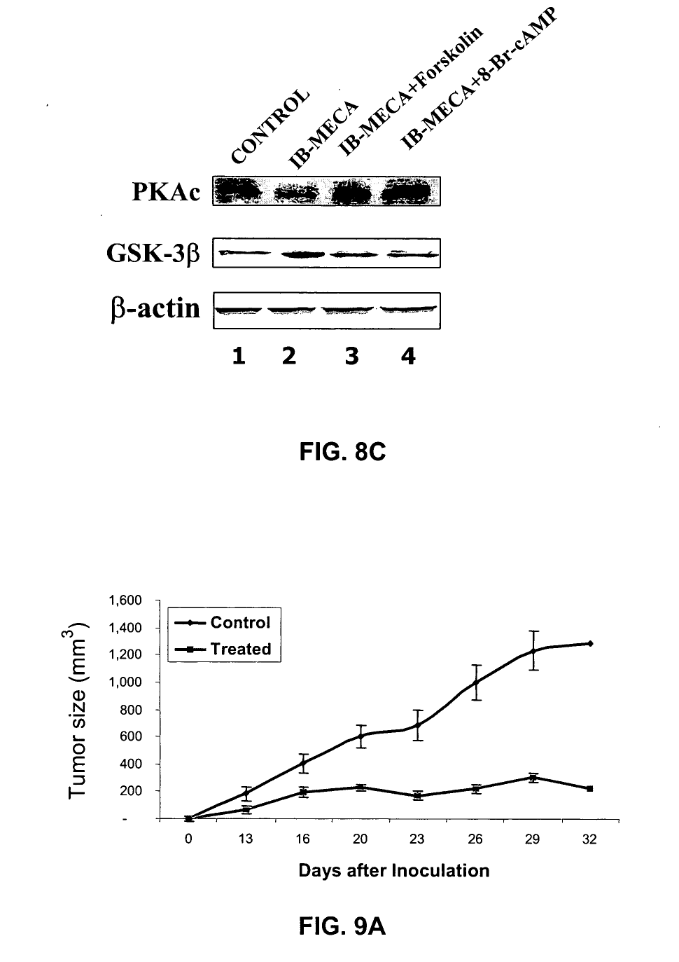

To test receptor functionality, the protein expression level of PKA and GSK-3β, which are modulated downstream to A3AR activation, was examined. Functional receptor sensitization by IB-MECA was observed after 15 and 60 minutes and was manifested by decreased PKA and increased GSK-3β levels. However, at 30 minutes, the PKA level stabilized and GSK-3β only slightly increased, indicating that receptor de-sensitization/re-sensitization took place upon chronic exposure to the agonist. The specificity of this response was demonstrated by introducing MRS1523, forskolin and 8-Br-cAMP, all known to counteract A3AR activation. Indeed, the modulatory effect of IB-MECA on PKA and GSK-3β was reversed in the presence of the above agents (FIG. 8a, 8b and 8c).

### Example 8

IB-MECA Inhibits Colon Carcinoma Development in Mice and Down-Regulates Expression of Biological Markers

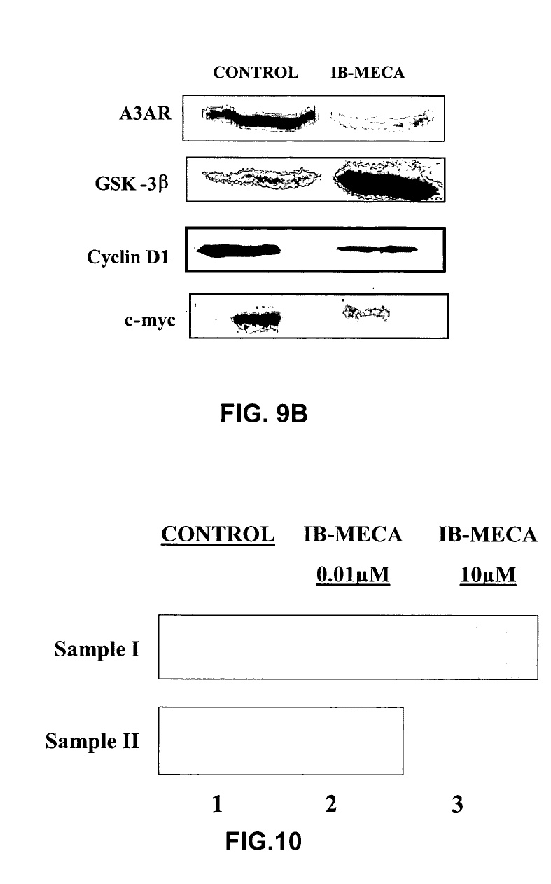

IB-MECA markedly suppressed the development of colon carcinoma cells in mice inoculated with HCT-116 human colon carcinoma cells (FIG. 9a). In tumor lesions excised from mice inoculated with the HCT-116 human colon carcinoma cell, Western blot analysis revealed down-regulation of A3AR, up-regulation of GSK-3β expression level, followed by a decrease in the level of c-myc and cyclin D1 (FIG. 9b). The level of the housekeeping protein β-actin did not change (data not shown).

### Example 9

Monitoring the Expression Level of Biological Markers in Clinical Treatment

The purpose of the following study is to determine the ability of IB-MECA at varying doses to alter the expression profile of relevant biological markers in subjects with newly diagnosed colorectal cancer. The subjects will be tested for the level of mRNA of the following markers prior to treatment by performing RT-PCR on tissue obtained in the diagnostic biopsy.

At least one, but preferably several of the following tumor markers are determined: A3AR receptor, PKA, PKB/Akt, GSK-3β, β-catenin, Cyclin D1, c-myc, NF-κB.

Subjects diagnosed include patients with colorectal lesions felt to have high likelihood of being malignant and who will most likely undergo biopsy followed by definitive surgery.

Biopsy specimens are removed from subjects (through colonoscopy) and undergo northern separation or RT-PCR amplification prior to treatment for testing for the above biological markers.

Treatment regimen: Cohorts of 5 to 10 patients are treated at escalating doses of IB-MECA, either daily or twice a day. Treatment with IB-MECA is initiated before definitive surgery.

After a set time of treatment with IB-MECA (a time which was previously determined to show maximal difference between the treatment and control) tumor lesion is removed at surgery and A3 receptor mRNA expression level along with downstream signals, PKA, PKB/Akt, GSK-3β, β-catenin. Cyclin D1, c-myc, PI3K, IKK, NF-κB, is determined by using RT-PCR. The level of expression of the different proteins will be compared to that determined from the biopsy specimen prior to treatment.

In the same manner, the effect of treatment with IB-MECA, via determination of the level of regulatory markers, is determined in cases of breast cancer, prostate cancer, melanoma and others. In a similar manner, mutatis mutandis, the effect of treatment in autoimmune inflammatory diseases may be determined.

### Example 10

Detection of A3AR Receptor on Human Neutrophils

10×10⁶ neutrophil cells isolated from 20 ml of human blood were incubated for 15 minutes with 0.01 mM or 10 mM of CF101 at 37° C. The cells were collected by centrifugation and washed with PBS. RNA was extracted from the cells by using TRI-reagent (Sigma). RNA level was quantified using spectrophotometer and 1 mg from each sample were subjected to RT-PCR using SuperScript One Step RT-PCR with Platinum Taq (Invitrogene), as described above in section E., by using the set No II as primers for amplification of 361 bp fragment. RT-PCR products were detected by electrophoresis and the size was verified by comparing with known RNA.

The results are shown in FIG. 10. As can be seen, the A3AR agonist IB-MECA was able to increase the expression of ARAR in neutrophils. This indicates that the neutrophils responded, in a pro-proliferative manner, to the therapeutic treatment. Therefore, detecting changes in the level of A3AR can indicate effectiveness of a treatment (by an A3AR agonist) for increasing neutrophil count, for example to counteract the effect of chemotherapy.

### Example 11

Modulation of Inflammatory Response and Expression of A3AR and Some Downstream Proteins by IB-MECA

Adjuvant induced arthritis (AIA) was induced in rats as described above. Rats were treated orally twice daily with either (i) vehicle (these rats serving as control), (ii) 10 μg/kg IB-MECA or with (ii) the specific A3AR agonist MRS 1220 followed 30 minutes later by IB-MECA. The mice were sacrificed on day 28 for histological scoring and measurement of signaling proteins and TNF-α.

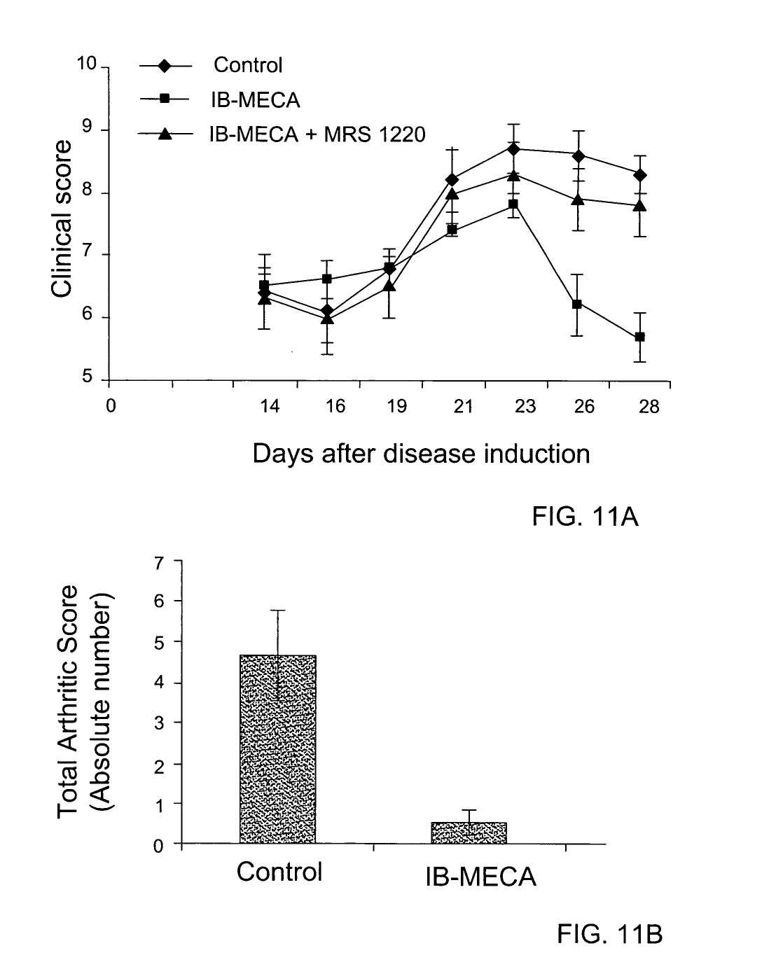

As can be seen in FIG. 11a, IB-MECA induced an anti-inflammatory response that was specific as it was nullified by the A3AR antagonist MRS 1220. The anti-inflammatory activity of IB-MECA can also be seen in the histological score (FIG. 11b)

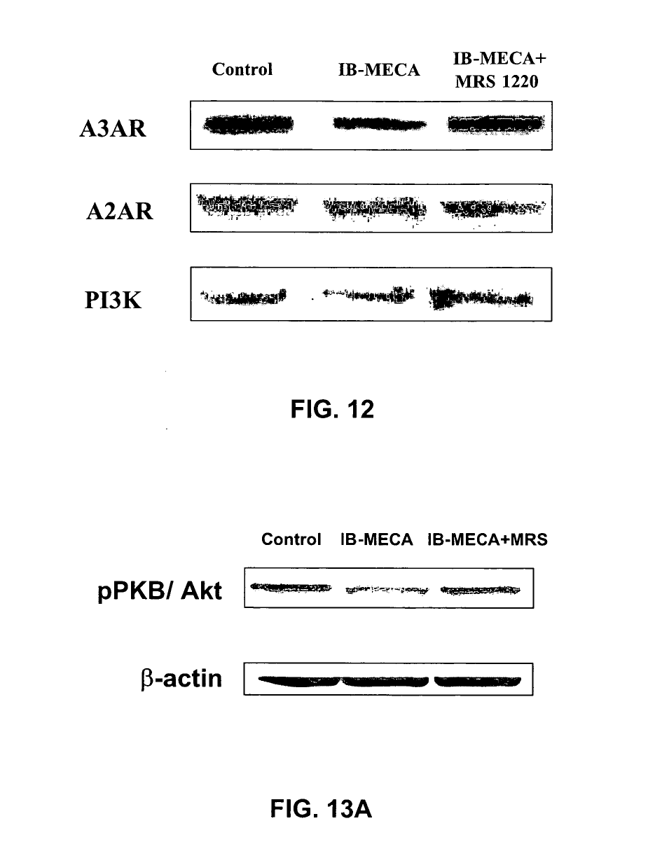

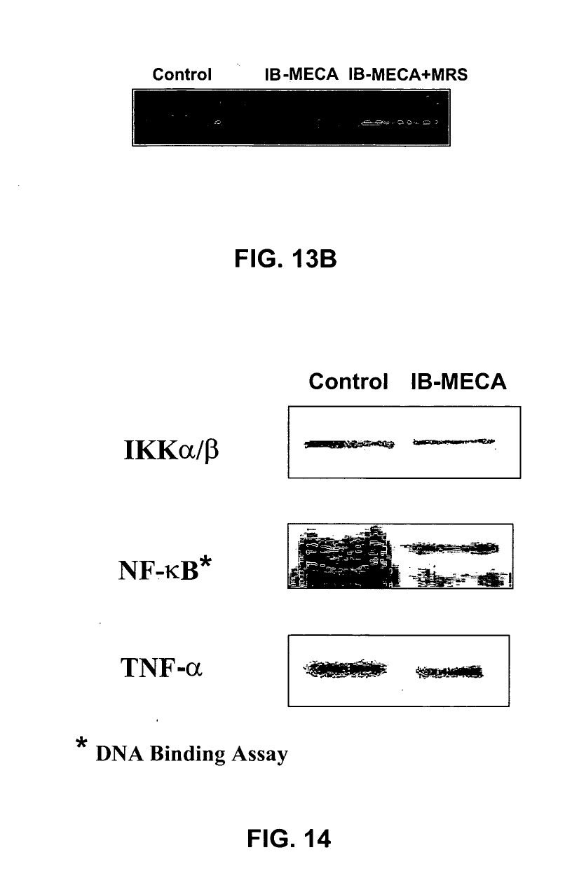

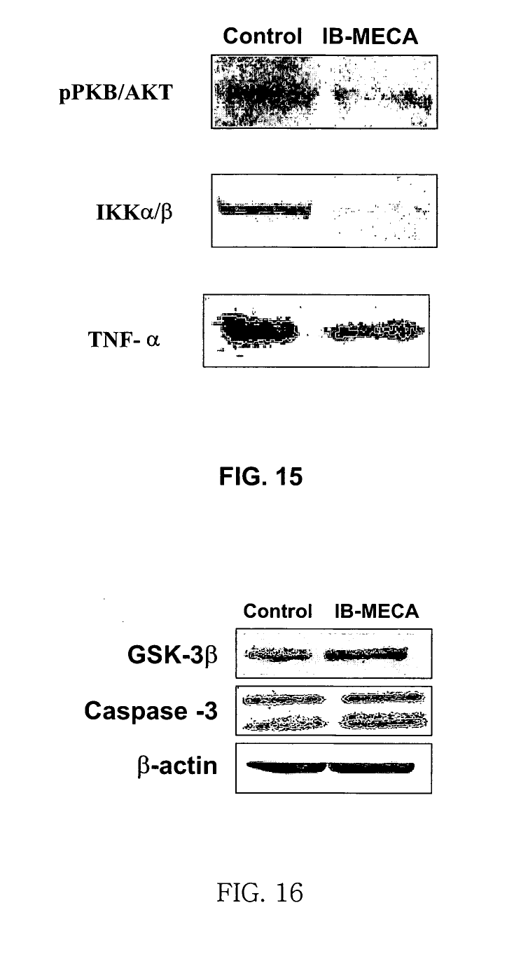

As can be seen in FIG. 12, the anti-inflammatory activity of IB-MECA was correlated with a down-regulation in the level of the A3AR, while having no effect on the A₂ adenosine receptor (A2AR). The MRS 1220 blocked this modulation demonstrating that it is specific and mediated through the A3AR. A similar down-regulation can be seen also in the downstream signaling proteins PI3K (FIG. 12), PKB/Akt (FIGS. 13a and 13b), IKKα/β NF-κB and TNF-α (FIGS. 14 and 15 for DLN and synovial tissue, respectively). An up-regulation is seen in the downstream signaling proteins including GSK-3β and caspase-3.

By monitoring the level of these signaling proteins, both in term of the expression as well as in terms of their activity, the effect of an A3AR agonist in the treatment of autoimmune inflammatory diseases can be assessed.

References

Olah et al, “The role of receptor structure in determining adenosine receptor activity”, Pharmacol Ther 85:55-75 (2000);

Poulsen et al, “Adenosine receptors: new opportunities for future drugs”, Bioorg Med Chem 6:619-641 (1998);

Fang et al, “Phosphorylation and inactivation of glycogen synthase kinase 3 by protein kinase A.”, Proc Natl Acad Sci USA 97:11960-11965 (2000);

Fishman et al, “Involvement of Wnt Signaling Pathway in IB-MECA Mediated Suppression of Melanoma Cells”, Oncogene 21:4060-4064 (2002);

Ferkey et al, “GSK-3: New Thoughts on an Old Enzyme”, Dev Biol, 225:471-479 (2000);

Bonvini et al, “Nuclear beta-catenin displays GSK-3beta- and APC-independent proteasome sensitivity in melanoma cells”, Biochim Biophys Acta 1495:308-318 (2000);

What is claimed is:

1. A method for monitoring the effectiveness of an administered agent that interacts with the A₃ adenosine receptor (A3AR) in a treatment of a disease state in an individual, the method comprising
(i) at a defined time point following administration of the agent to the individual, selected such so as to permit the agent to reach and affect cells in the individual that are associated with the disease state, withdrawing a sample of said or tissue containing said cells from the;
(ii) detecting the level of at least one physiological parameter of at least one biological marker in said cells, the marker being an A3AR,or an element associated with the A3AR signal transudation pathway downstream to A3AR; and
(iii) comparing the level of said at least one parameter to a control level, being the level thereof in such cells or tissue from the same individual before administration of said agent, or being a standard reference for said marker which is indicative of a n un treated disease state;
wherein a difference in level of the physiological parameter from control being indicative of the effectiveness of said treatment against these state.

2. The method according to claim 1 wherein the agent that interacts with the A3AR is an A3AR agonist.

3. A method according to claim 1 wherein the A3AR signal transduction pathway is the Wnt pathway.

4. A method according to claim 3 wherein the element is at least one element selected from: PKA, PKB/Akt, GSK-3β, β-catenin, cyclin D1, c-myc.

5. A method according to claim 1 wherein the A3AR signal transduction pathway is the NF-κB pathway.

6. A method according to claim 5 wherein the element is at least one element selected from: NF-κB, PI3K, IKK, c-myc, cyclin D1.

7. A method according to claim 1 wherein the physiological parameter is selected from: the level of mRNA or protein expression, the level of phosphorylation and the cellular localization.

8. The method of claim 1, wherein said disease state is a proliferative-related disease.

9. The method of claim 8, wherein said disease is cancer.

10. The method of claim 9, wherein said cancer is melanoma, colon carcinoma or prostate cancer.

11. The method of claim 8, wherein said disease is an inflammatory disease.

12. The method according to claim 8, wherein effective treatment against the disease is indicated by a change in a physiological parameter of a biological marker selected from:
(a) a decrease of the protein level or the mRNA level coding therefore of at least one of A3AR, PKB/Akt, PKA, β-catenin, c-myc, cyclin D1 and NF-κB, TNF-α; or increase in the protein level or mRNA coding therefore of GSK-3β
(b) at least one change in phosphorylation level selected from: decrease in phosphorylation level of GSK-3β, increase in the phosphorylation level of PKB/Akt, PKA or β-catenin.
(c) at least one change in cellular localization selected from: decrease in the localization of A3AR receptor in the cellular membrane as compared to control, decrease in the localization of β-catenin or NF-κB in the nucleus as compared to cytosol.

13. A method according to claim 1, wherein said disease state is a disease or condition wherein a beneficial therapeutic effect is evident by increase proliferation.

14. The method of claim 13, wherein said disease state is a decrease in white blood cell count, especially neutrophils as a result of chemo- or radio-therapy.

15. The method of claim 13, wherein effective treatment against the disease is indicated by a change in a physiological parameter of a biological marker selected from:
(a) increase of the protein level or of the level of mRNA coding therefore of at least one of A3AR, PKB/Akt, PKA, β-catenin, c-myc, cyclin D1 and NF-κB, or decrease in the protein or mRNA level of GSK-3β;
(b) at least one change in phosphorylation level selected from: increase in phosphorylation level of GSK-3β, decrease in the phosphorylation level of PKB/Akt, PKA or in the phosphorylation level of β-catenin.
(c) at least one change in cellular localization selected from: increase in the localization of A3AR receptor in the cellular membrane as compared to control, increase in the localization of β-catenin in the nucleus as compared to cytosol.

16. A method according to claim 1, wherein the level of the at least one physiological parameter of the at lease one biological marker is determined at a time point after the administration of the agent, wherein the differences between the level of the parameter in the treated subject and the untreated control are expected to be the most prominent.

17. A method according to claim 2 wherein the A3AR agonist is 1-deoxy-1-[6[[(3-iodophenyl)methyl]amino]-9H-purine-9-yl]-N-methyl-β-D-ribofura-nuronaminde (IB-MECA).

18. A method for determining whether a drug candidate is an A3AR agonist useful in treating a disease state manifested in diseased cells, the method comprising:
(i) obtaining a sample of the diseased cells or tissue containing said cells;
(ii) contacting said sample with the drug candidate;
(iii) detecting the level of at least one physiological parameter of at least one biological marker in said cells, the marker being an A3AR, or an element associated with the A3AR signal transudation pathway which is downstream to the A3AR; and
(iv) comparing the level of said at least one parameter to the level thereof in untreated sample not contacted with said drug candidate;
wherein a difference in level of the physiological parameter between the treated and untreated sample being indicative that the drug candidate is an agonist of A3AR.

19. A method according to claim 18 wherein the A3AR signal transduction pathway is the Wnt pathway.

20. A method according to claim 19, wherein the element is at least one element selected from: PKA, PKB/Akt, GSK-3β, β-catenin, cyclin D1, c-myc.

21. A method according to claim 18 wherein the A3AR signal transduction pathway is the NF-κB pathway.

22. A method according to claim 21 wherein the element is at least one element selected from: NF-κB, PI3K, IKK, TNF-α, c-myc, cyclin D1.

23. A method according to claim 18 wherein the physiological parameter is selected from: the level of mRNA or protein expression, the level of phosphorylation and the cellular localization.

24. The method of claim 18 wherein said disease state is a proliferative-related disease.

25. A method for determining whether a drug candidate is an A3AR agonist useful in treating a disease state manifested in diseased cells, the method comprising:
(i) administering said drug candidate to a subject having said disease state;
(ii) at one or more defined time points following the administration, withdrawing a sample of the diseased cells or tissue containing said cells from the subject;
(iii) detecting the level of at least one physiological parameter of at least one biological marker in said cells, the marker being an A3AR, or an element associated with the A3AR signal transudation pathway which is downstream to the A3AR; and
(iv) comparing the level of said at least one parameter to the level in diseased cells withdraw from a subject not administered with said drug candidate;
wherein a difference in level of the physiological parameter between the treated and untreated sample being indicative that the drug candidate is an agonist of A3AR.

26. A method according to claim 25 wherein the A3AR signal transduction pathway is the Wnt pathway.

27. A method according to claim 26, wherein the element is at least one element selected from: PKA, PKB/Akt, GSK-3β, β-catenin, cyclin D1, c-myc.

28. A method according to claim 25 wherein the A3AR signal transduction pathway is the NF-κB pathway.

29. A method according to claim 22 wherein the element is at least one element selected from: NF-κB, PI3K, IKK, TNF-α, c-myc, cyclin D1.

30. A method according to claim 25 wherein the physiological parameter is selected from: the level of mRNA or protein expression, the level of phosphorylation and the cellular localization.

31. The method of claim 25 wherein said disease state is a proliferative-related disease.
